(19) United States
(12) Patent Application Publication
(10) Pub. No.: US 2026/0272719 A1
(43) Pub. Date: Sep. 17, 2026

(54) A Non-Adherent Absorbent Article

(71) Applicant: Serigen Mediproducts Pvt. Ltd., Pune, Maharashtra (IN)

(72) Inventors: Raeesa SAYYAD, Thane, Maharashtra (IN); Lakshmi Raghavan PILLAI, Ernakulam, Kerala (IN); Nimisha PAREKH, Pune, Maharashtra (IN); Swati Govind SHUKLA, Pune, Maharashtra (IN); Anuya NISAL, Pune, Maharashtra (IN); Rucha DESHPANDE, Pune, Maharashtra (IN)

(21) Appl. No.: 19/163,004

(22) PCT Filed: Jul. 1, 2024
(86) PCT No.: PCT/IN2024/051008

§ 371 Date: Sep. 8, 2025

(30) Foreign Application Priority Data
Jun. 29, 2023 (IN) 202321043599

(51) Int. Cl. A61F 13/01; A61F 13/00

(52) U.S. Cl. CPC A61F 13/01046; A61F 13/00063; A61F 2013/00229

(57) ABSTRACT

The present invention relates to a non-adherent, absorbent article with an optimum moisture vapor transmission rate (MVTR) that may be used as wound dressing or as an anti-adhesive membrane in surgical devices. The present invention further relates to an article comprising of a polymer deposited/coated on to a woven and/or non-woven mesh and to the process of fabrication thereof.

### FIELD OF THE INVENTION

The present invention relates to a non-adherent, absorbent article with an optimum moisture vapor transmission rate (MVTR) that may be used as wound dressing or as an anti-adhesive membrane in surgical devices. The present invention further relates to an article comprising of a polymer deposited/coated on to a woven and/or non-woven mesh and to the process of fabrication thereof.

### BACKGROUND OF THE INVENTION

Wound healing is a complex and dynamic process that includes various stages like haemostasis, inflammation, proliferation, and re-modelling. These stages work in an orchestrated manner to repair damaged tissue and restore function. Factors such as infection, poor blood supply, or underlying medical conditions can further impair the normal progression of wound healing.

Several commercial dressings have been launched that cater to various aspects of wound healing. Cotton gauze-based dressings have a higher absorption capacity, but lack the important characteristic of being non-adherent. Vaseline coated cotton gauzes are non-adherent, but exhibit poor absorption capacity and sub-optimal moisture vapor transmission rate (MVTR). Thus, there is a need to develop a wound dressing that combines all these essential properties of non-adherence, absorption and appropriate MVTR. Lack of this combination of properties impairs the wound healing process. The poor performance of wound dressings also results in discomfort to the patient and adds to the recuperation time.

In recent times, silk-based proteins have shown promising results in wound healing applications.

U.S. Pat. No. 6,175,053 discloses a wound dressing material, which comprises of an amorphous film of crystallinity below 10% containing Fibroin and Sericin as a main component.

US20210252182 discloses a composite dressing comprising of a combination of one layer of porous matrix composed of biomaterial and at least one layer of fabric support. Wherein the biomaterial can be chitosan, alginates, starch, cellulose, gelatin, collagen, polyethylene glycol (PEG), polyvinyl alcohol (PVA), polyurethanes, hyaluronic acid, poly-lysine, poly-glutamic acid, poly(ethylene oxide) (PEO), poly(propylene oxide) (PPO), poly(acrylic acid), poly(methacrylic acid) and their derivatives or any combination thereof. The fabric support is prepared from natural material selected from a group comprising cotton, wool, silk and blends or synthetic material selected from a group comprising polyamide, polyester, rayon and blends thereof.

U.S. Pat. No. 8,728,498 discloses electrospun silk material systems for wound healing comprising of polyethylene oxide (PEO) and aqueous silk fibroin (SF) solution.

WO2004001103 discloses SF with PEO in the ratio of 80:20 and 70:30 as a wound healing blend.

Article titled “SF and silk-based biomaterial derivatives for ideal wound dressings” DOI 10.1016/j.ijbiomac.2020.08.041 discloses SF based composite films using different types of polymers such as PEG, PVA, alginate, and hyaluronic acid for wound healing.

“Fabrication of Chitosan/Silk Fibroin Composite Nanofibers for Wound-dressing Applications” by Zeng-xiao Cai et al. published in Int. J. Mol. Sci. 2010, 11(9), 3529-3539; discloses composite nanofibrous membranes of chitosan (CS) and SF with following ratios: CS/SF (80/20); (b)CS/SF (50/50); (c) CS/SF(20/80) for various biomedical applications.

The prior arts suggests that the desirable characteristics of non-adherence, absorption and optimum MVTR, is a need in the art. Further, an article with these characteristics can also be used as a non-adherence barrier during surgeries.

### SUMMARY OF THE INVENTION

It is therefore an object of the present invention to provide a non-adherent, absorbent wound healing article with optimum MVTR and to the process of preparation thereof.

The other objective is to provide a non-adherent article, which has excellent absorption of exudate.

In an aspect, the present invention provides a non-adherent, absorbent wound healing article comprising;
i. A first contact layer of regenerated silk fibroin (SF) containing the hydrophilic natural and/or synthetic polymer coated/deposited on to the mesh; and
ii. Optionally loaded with bioactive molecules.

Hydrophilic polymers used herein are those polymers which dissolve in water or are swollen by water. These polymers also typically exhibit a surface contact angle <90 degree.

The article of the present invention has a thickness of 1-20 mm and a density of 0.02-0.08 g/cm³.

The regenerated SF in the first contact layer is in the concentration ranging between 0.25-8 wt %, preferably 1-4 wt %; the hydrophilic synthetic or natural polymer is in the concentration ranging between 0.25-8 wt %, preferably 1-4 wt %; and wherein the SF to hydrophilic natural or synthetic polymer are in the ratio ranging between 95:05 to 50:50.

The hydrophilic synthetic polymer is selected from water soluble polymer such as polyethylene glycol, polyethylene oxide, polyvinyl alcohol and the like alone or in combination thereof.

The hydrophilic natural polymer is selected from water soluble polymers such as collagen, gelatin, chitosan, alginate, sericin, hyaluronic acid and such like alone or in combination thereof.

The bioactive molecule is selected from pharmaceutical drugs, nanoparticles, ions, natural plant/herbal extracts, phytochemicals, growth factors, healing agents, disinfectants, astringents, regenerating agents and such like alone or combination thereof in an amount of 0.01 to 5 mg/ml.

In another aspect, the present invention provides a non-adherent, absorbent wound healing article with optimum moisture vapor transmission rate (MVTR) comprising;
i. Regenerated silk fibroin (SF) along with hydrophilic synthetic and/or natural polymer coated/deposited on to the mesh;
ii. Optionally loaded with bioactive molecules in an amount of 0.01 to 5 mg/ml;
wherein said SF to hydrophilic polymer has a ratio ranging between 95:05 to 50:50.

In an aspect, the regenerated SF (prepared from natural silk cocoons) has concentrations ranging from 0.25 to 8 wt %, most preferably in the range 1-4 wt %.

The hydrophilic synthetic polymer is selected from water soluble polymer such as polyethylene glycol, polyethylene oxide, polyvinyl alcohol and such like in an amount of 0.25-8 wt %.

In another aspect, the hydrophilic natural polymer is selected from water soluble polymers such as collagen, chitosan, alginate, sericin, hyaluronic acid and such like in an amount of 0.25-8 wt %.

The bioactive molecule is selected from pharmaceutical drugs, nanoparticles, growth factors, healing agents, disinfectants, astringents, regenerating agents, natural plant/herbal extracts, ions, phytochemicals and such like alone or combinations thereof in an amount of 0.01 to 5 mg/ml such that the addition of the bioactive molecules do not adversely affect the essential properties of the article.

In another aspect, the present invention provides a process for preparing the article comprising;
i. Preparing the regenerated silk fibroin (SF) solution;
ii. Mixing the regenerated SF solution with hydrophilic synthetic and/or natural polymers in all possible combinations in the ratio ranging between 95:05 to 50:50 to obtain the polymer solution;
iii. Placing the mesh in to the mold;
iv. Pouring the required amount of the polymer solution of step (ii) in to the mold and freezing followed by lyophilization;
v. Annealing the lyophilized product with lower alcohol.

In an aspect, the silk content in the regenerated silk fibroin is 65-95%.

The mesh of the present invention may be a woven and/or non-woven mesh.

In another aspect, the polymer solution of the present process may be loaded with the bioactive molecule selected from pharmaceutical drugs, nanoparticles, growth factors, healing agents, disinfectants, astringents, regenerating agents, natural plant/herbal extracts, ions, phytochemicals and such like alone or combinations thereof. The amount of the bioactive molecule will depend on the particular drug being employed and medical condition being treated. Preferably, the bioactive molecule is loaded in an amount of 0.01 to 5 mg/ml.

The article of the present invention can be removed physically without any pain and without disturbing the top healing surface of the wound.

In an aspect, the article of the present invention exhibits the unique non-adherent property which facilitate superior and faster healing since removal of said article does not disturb the top healing surface as demonstrated by the in-vitro study below.

In an aspect, the article of the present invention exhibits excellent absorption of the exudate.

In an aspect, the article of the present invention is used for wound healing.

In another aspect, the article of the present invention is used as an anti-adhesive membrane in surgical devices,

In another aspect, the present invention provides a method of promoting the wound healing comprising contacting the wound with the said article optionally containing the bioactive compound.

In another aspect, the present invention provides a non-adhesive, absorbent article that has optimum MVTR and hence facilitates accelerated healing of wounds when used for wound dressing.

In yet another aspect, the non-adhesive, absorbent article of the present invention acts as an anti-adhesion membrane with desired properties with drugs incorporated into the membrane, so that the drug can be delivered locally over a period of time to the surgical site.

The non-adhesive, absorbent article of the present invention has good handling characteristics when used for wound dressings as well as during a surgical procedure, is compatible to the tissues, is resorbable and possesses sufficient bioadhesiveness to ensure secure placement at the surgical site until the likelihood of adhesion formation is minimized.

In t another aspect, the present invention provides a non-adherent, absorbent wound dressing composition with optimum moisture vapor transmission rate (MVTR) comprising;
i. Regenerated SF along with Hydrophilic synthetic and/or natural polymer coated/deposited on to the mesh; and
ii. optionally loaded with bioactive molecules in an amount of 0.01 to 5 mg/ml;
   wherein said SF to hydrophilic polymer is in the ratio ranging between 95:05 to 50:50.

### DESCRIPTION OF THE FIGURES

FIG. 1: Depict the non-adherent property of the present article.

FIG. 2: Depict the percentage absorption of the present article wherein the percentage absorption is higher when lower polymer solution is used.

FIG. 3: (A) Depict the percentage absorption at different mixing ratios of solution wherein higher absorption is observed for SF:HP ratio of 75:25; (B) Percent water absorption with varying the number of layers of cotton gauze (CG) with silk blended with hydrophilic polymer.

FIG. 4: (A) Depict the moisture vapor transmission rate (MVTR) with different concentration of polymer blends; (B) Normalized MVTR with various layer of cotton gauze (CG); (C) Normalized MVTR with different woven and non-woven matrix with silk blended with hydrophilic polymer.

FIG. 5: Loading of the antibiotic in the article and the amount release.

FIG. 6: In vivo wound healing studies in rat wound healing model. (A) Photographical evidence of wound healing in rats treated with cotton gauze, and the present article on day 0 and day 15.

### DETAILED DESCRIPTION OF THE INVENTION

The invention will now be described in detail in its various preferred and optional embodiments so that various aspects of the invention will be more clearly understood, however, do not limit the scope of the invention.

### Geographical Origin and Source of the Biological Material:

Silk Fibroin (SF) solution is extracted from the Bombyx mori pure bivoltine cocoons sourced from Pappini Amman Silks, Tamilnadu.

Unless defined otherwise, all technical and scientific terms used herein have the same meaning as those commonly understood to one of ordinary skill in the art to which this invention pertains.

The unique feature of the present invention is that the article provides the characteristic feature of non-adherence as they reduce the discomfort and pain for patient during dressing change. Further, non-adherent dressings also facilitate superior and faster healing because removal of dressing does not disturb the top healing surface. Further the dressings also absorb the exudate from the wound and provide an optimum moisture vapor transmission rate that accelerates the rate of wound healing. The same is achieved in the present invention by providing a non-adherent, absorbent article having low concentration of the polymer coated/deposited on to the mesh.

The term “hydrophilic polymers” means both synthetic and natural polymers which dissolve in or are swollen by water or biofluids without dissolving, are biocompatible.

The term “biocompatible” means the polymer of the present invention is not harmful or toxic to living tissues.

In an embodiment, the present invention relates to a non-adherent, absorbent article comprising;
i. A first contact layer of natural silk fibroin (SF) containing the hydrophilic natural and/or synthetic polymer coated/deposited on to the mesh; and
ii. Optionally loaded with bioactive molecules;
   wherein the SF to hydrophilic polymer are in the ratio ranging between 95:05 to 50:50.

The article of the present invention has a thickness of 1-20 mm, preferably of 1-8 mm and density of 0.02-0.08 g/cm³.

The regenerated SF solution is in the concentration ranging between 0.25-8 wt %, preferably 1-4 wt %; the hydrophilic synthetic or natural polymer is in the concentration ranging between 0.25-8 wt %, preferably 1-4 wt %.

The hydrophilic synthetic polymer is selected from water soluble polymer such as Polyethylene glycol, polyethylene oxide, polyvinyl alcohol and the like alone or in combination thereof.

The hydrophilic natural polymer is selected from water soluble polymers such as collagen, gelatin, chitosan, alginate, sericin, hyaluronic acid and such like alone or in combination thereof.

The bioactive molecule is selected from pharmaceutical drugs, nanoparticles, growth factors, healing agents, disinfectants, astringents, regenerating agents, natural plant/herbal extracts, ions, phytochemicals and such like alone or combinations thereof in an amount of 0.01 to 5 mg/ml.

The article can be removed physically without any pain and without disturbing the top healing surface.

In another embodiment, the present invention relates to a non-adherent absorbent article with optimum MVTR comprising;
i. Regenerated SF along with Hydrophilic synthetic and/or natural polymer coated/deposited on to the mesh;
ii. Optionally loaded with bioactive molecules in an amount of 0.01 to 5 mg/ml;
   wherein said SF to hydrophilic polymer has a ratio ranging between 95:05 to 50:50.

The mesh of the present invention is a woven and/or non-woven mesh and is made of the material selected from cotton, silk, cellulose, nylon, polyester and such like.

In an embodiment, the regenerated silk fiboin (SF) is in the concentration ranging between 0.25-8 wt %, preferably in the range of 1-4 wt %.

In another embodiment, the hydrophilic synthetic polymer is selected from water soluble polymer such as polyethylene glycol, polyethylene oxide, polyvinyl alcohol and the like alone or in combination thereof in an amount of 0.25-8 wt % and most preferably 1-4 wt %.

In yet another embodiment, the hydrophilic natural polymers is selected from water soluble polymers such as collagen, gelatin, chitosan, alginate, sericin, hyaluronic acid and such like alone or in combination thereof in an amount of 0.25-8 wt % and most preferably 1-4 wt %.

The bioactive molecule is selected from pharmaceutical drugs, nanoparticles, growth factors, healing agents, disinfectants, astringents, regenerating agents, natural plant/herbal extracts, ions, phytochemicals and such like alone or combinations thereof in an amount of 0.01 to 5 mg/ml such that the addition of the bioactive molecules do not adversely affect the essential properties of the article.

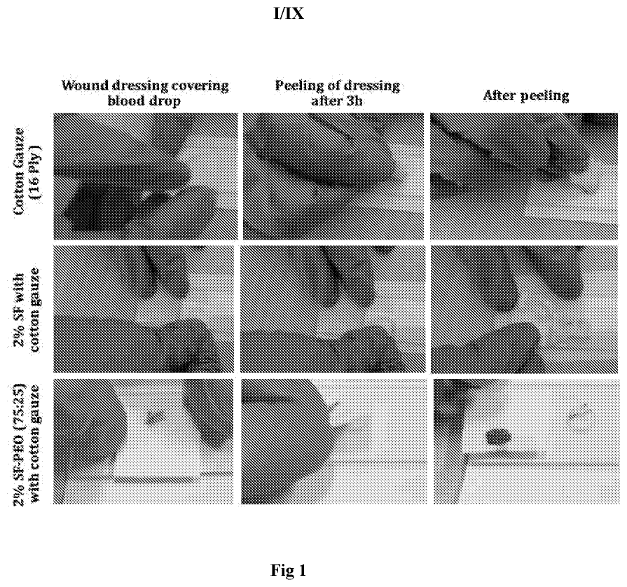

In an embodiment, the article of the present invention comprising of said components remains in position and can be removed painlessly and easily without applying any pressure or without requiring saturating it with saline, water or physiological buffer as indicated in the FIG. 1.

In another embodiment, the present invention relates to a process for fabricating the non-adherent, absorbent article comprising;
i. Preparing the regenerated silk fibroin (SF) solution;
ii. Mixing the regenerated SF solution with hydrophilic synthetic and/or natural polymer in the ratio ranging between 95:05 to 50:50 to obtain the polymer solution;
iii. Placing the mesh into the mold;
iv. Pouring the required amount of the polymer solution of step (ii) in to the mold and freezing followed by lyophilization;
v. Annealing the lyophilized product with lower alcohol.

Accordingly, the regenerated SF solution was prepared by dissolution of the fibroin in Lithium Bromide (LiBr) followed by dialysis. The fibroin was obtained from Bombyx mori pure bivoltine cocoons. The regenerated SF solution with the concentration in the range of 5-6 wt % was diluted with deionized water to obtain the SF in the concentration of 1-4 wt %. Further, the desired concentrations of aqueous solutions of either synthetic or natural hydrophilic polymers were prepared by dissolving said polymer in DI water at room temperature. The hydrophilic polymer solution was mixed preferentially with the same concentration of SF solutions in volume ratios of 05:95 to 50:50 and poured into the mold in which a woven and/or non-woven mesh was kept. This was followed by freezing at −80° C. and lyophilized for 12-18 h followed by exposure to lower alcohol such as methanol. The annealing of the product with lower alcohol enabled the conformation transition in SF protein and improved their stability.

In another embodiment, the polymer solution of the present process may be loaded with the bioactive molecule selected from pharmaceutical drugs, nanoparticles, growth factors, healing agents, disinfectants, astringents, regenerating agents, natural plant/herb extracts, ions, phytochemicals and such like alone or combinations thereof in suitable concentration. The amount of the bioactive molecule will depend on the particular drug being employed and medical condition being treated. Preferably, the bioactive molecule is loaded in an amount of 0.01 to 5 mg/ml.

In yet another embodiment, the present invention relates to a non-adherent, absorbent article with optimum MVTR comprising;
i. Regenerated SF in the concentration of 2 wt % along with hydrophilic natural polymer selected from chitosan in concentration of 2 wt % coated/deposited on to the mesh;
ii. optionally loaded with bioactive molecules in an amount of 0.01 to 5 mg/ml;
   wherein said SF to chitosan is in the ratio of 75:25.

In another embodiment, the present invention relates to a non-adherent, absorbent article with optimum MVTR comprising;
i. Regenerated SF in the concentration of 2 wt % along with hydrophilic synthetic polymer selected from polyethylene oxide (PEO) in concentration of 2 wt % coated/deposited on to the mesh;
ii. optionally loaded with bioactive molecules in an amount of 0.01 to 5 mg/ml;
   wherein said SF to polyethylene oxide (PEO) is in the ratio of 75:25.

The bioactive molecule is selected from pharmaceutical drugs, nanoparticles, growth factors, healing agents, disinfectants, astringents, regenerating agents, natural plant/herb extracts, ions, phytochemicals and such like alone or combinations thereof in an amount of 0.01 to 5 mg/ml such that the addition of the bioactive molecules do not adversely affect the essential properties of the wound dressing material.

In an embodiment, the present invention disclose the non-adherent, absorbent article with optimum MVTR for use as wound dressing material for accelerated wound healing.

In an embodiment, the present invention disclose a method of promoting the wound healing comprising contacting the wound with said article optionally containing the bioactive compound.

In another embodiment, the physical properties of the present article were evaluated.

### (A) Water Absorption

The percentage of water absorption was studied by immersing the sample, with initial weight W1, cut into 1 cm×1 cm size piece in 2 ml of DI solution. After 2 h, the sample was removed and the final weight (W2) was recorded after gently tapping on tissue paper to remove excess dripping water. % water absorption was calculated by using following formula.

$$\% Water Absorption = ( W 2 - W 1 ) / W 1 \times 100$$

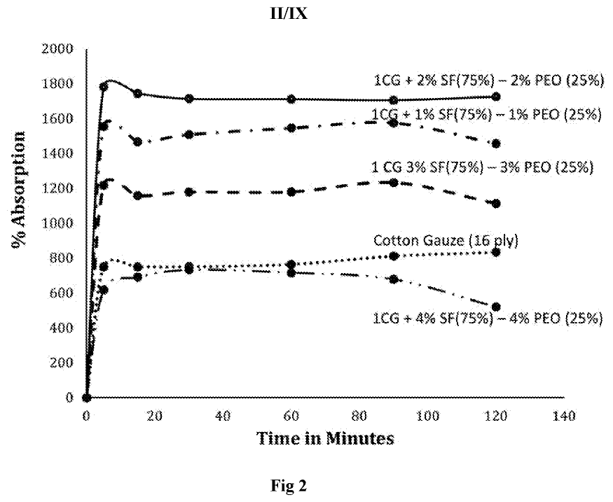

It has been observed from the FIG. 2 that % water absorption was higher when lower polymer solution concentration is used. The absorption was found to be higher for polymer concentrations between 1-3%, while as the concentration of polymer was increased to 4%, the absorption was found to be very low.

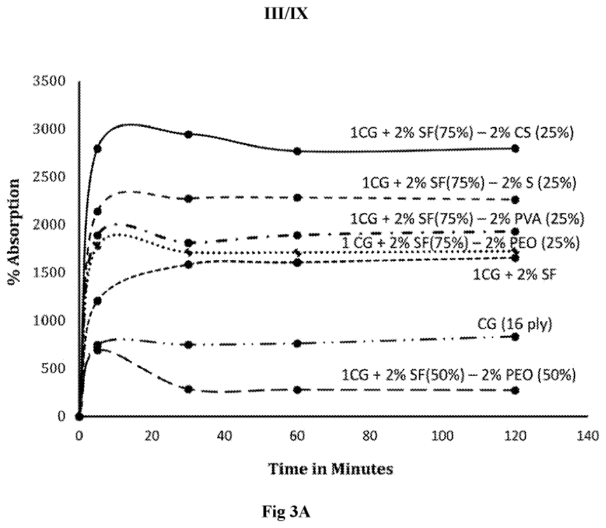

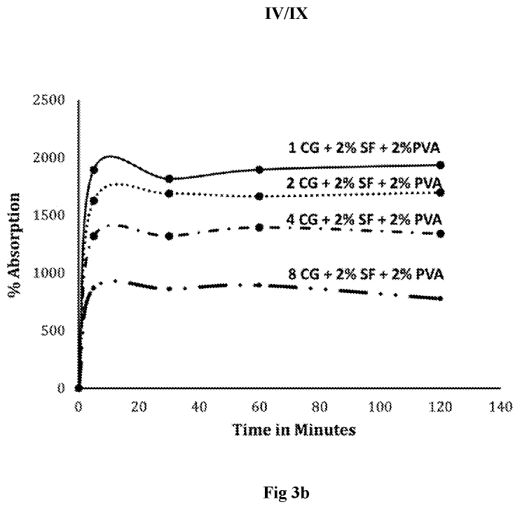

The percentage absorption was further studied with different mixing ratios of SF and hydrophilic polymers. It has been observed that % Absorption is higher for any hydrophilic polymer (1P), where ratio of SF:HP is 75:25 (FIG. 3A). However, with increase in thickness (layers) of cotton gauze (CG), the percent absorbance was observed to decrease as depicted in FIG. 3B.

### (B) Moisture Vapor Transmission Rate (MVTR)

MVTR is an important property that governs the performance of an article especially in wound healing applications. To measure the MVTR of developed wound dressings, a Paddington cup was used. The cup contains a mold with a holding capacity of 15-18 mL liquid and a lid. Weight of the assembly together with the base and lid was recorded (W1). Samples were kept on the surface in such a way that the sample must not come in contact with DI and tightly covered with upper closing lid of MVTR assemblies. Approximately 15 ml DI was added in the mold and the weight of the assembly was recorded (W2).

The assembly was placed in the incubator at 37° C. for 24 h. At the end of incubation, the assembly was removed and kept in the desiccator and allowed to equilibrate at room temperature for 30 minutes. Post this, the weight was recorded (W3). Further, the assembly was also reweighed after removing all the water (Assembly without water+Sample+lid) (W4).

The mass of moisture vapor lost through the dressings by the formula (W2-W3).

Calculate the MVTR by the formula MVTR (g/m²/24 h)=(W2-W3)/Area of sample.

Since, MVTR is highly influenced by thickness of the sample, resultant MVTR has been normalized with thickness and reported as g/m2/24 h/cm.

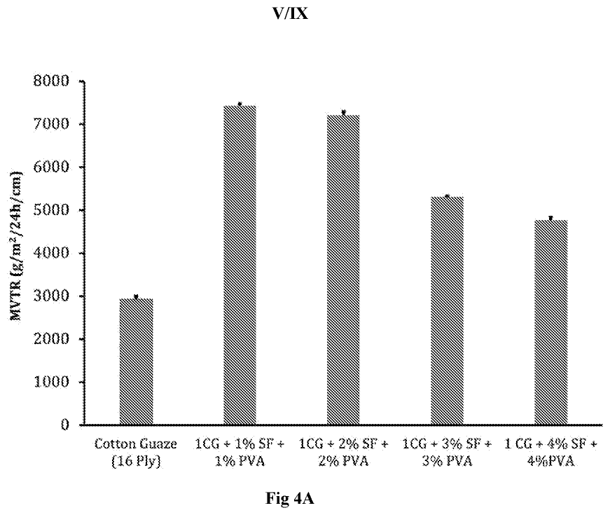

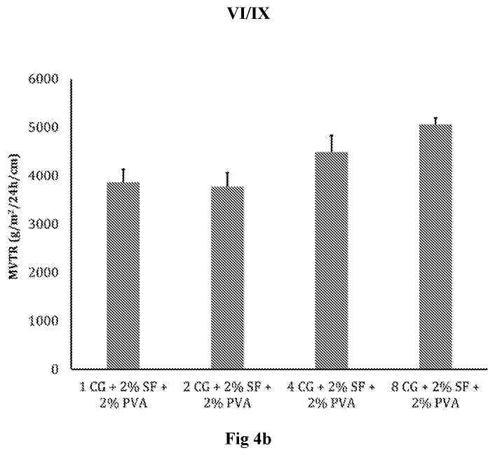

It was observed that addition of hydrophilic polymer to SF resulted in significant increase in MVTR (FIG. 4A). The MVTR rate was observed to increase with increase in layers of CG with silk blended with hydrophilic polymer as depicted in FIG. 4B. As depicted in FIG. 4C, it is observed that MVTR can be tailored as per the clinical condition of the wound by fabricating the article with varied components. The influence of various components on MVTR follows an unpredictable trend.

### (C) Non-Adherent Property

Non-adherent property in case of wound dressings are highly desirable as they reduce discomfort and pain for patient during dressing change. Further, non-adherent dressings also facilitate superior and faster healing because removal of dressing does not disturb the top healing surface. However, there are no standard protocols for testing non-adherence of dressings in in-vitro tests. The present inventors have developed and standardised a protocol to establish non-adherence which is not reported in the art hitherto.

Accordingly, a single blood drop was taken by finger prick method and placed on a clean glass slide. Immediately after collecting the blood drop, the developed article was kept on the glass slide in such a way that blood drop was covered completely. The samples were incubated for 3 h. After 3 h, the samples were peeled off from the surface of the glass slide which depicted non-adherence as shown in FIG. 1.

### (D) Loading of the Bioactive Drug

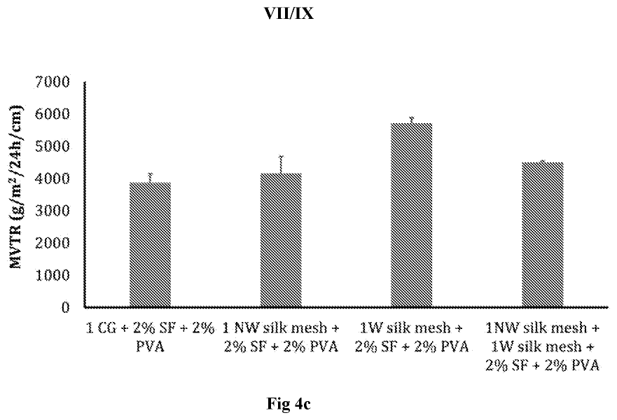

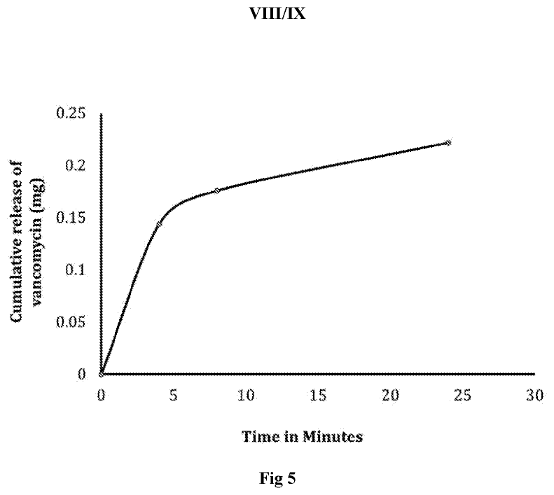

Vancomycin at the concentration of 0.5 mg/mL was loaded in the polymer solutions while preparing said article for wound dressings. Accordingly, the piece containing the drug was incubated in DI water 25° C. for 24 h. Release in DI was measured by UV spectroscopy by recording the absorbance at 280 nm. The absorbance value was used to calculate the actual concentration of drug released by comparing the absorbance with the standard curve. As shown in FIG. 5, the drug could be successfully incorporated and released from the said article. This proved that the process was flexible enough to incorporate various active biomolecules, drugs, growth factors, etc. into the said article.

### (E) In Vivo Wound Healing Studies in Rat Wound Healing Model.

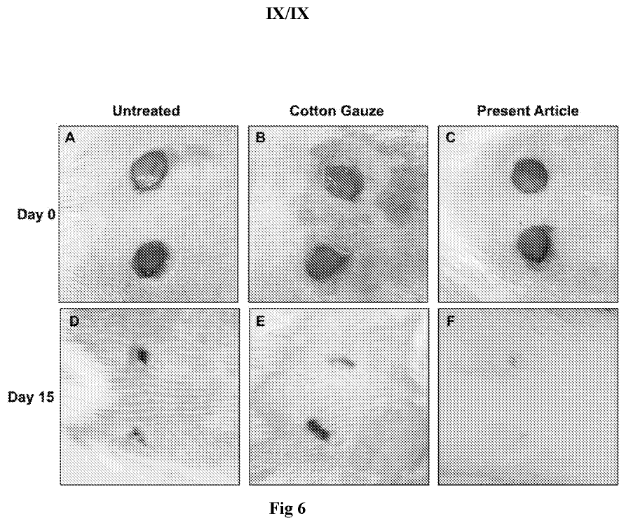

As depicted in FIG. 6(A-F), significant wound closure (98.9±0.3%) was observed in treated wistar rats as compared to the untreated wounds (96.4±0.3%) and cotton gauze treated (92.3±0.7%) wounds.

In an embodiment, the non-adherent, absorbent article of the present invention can be removed physically without any pain and without disturbing the top healing surface of the wound.

In an embodiment, the article of the present invention exhibits the unique non-adherent property which facilitate superior and faster healing since removal of said article does not disturb the top healing surface as demonstrated by the in-vitro study below.

In an embodiment, the article of the present invention exhibits excellent absorption of the exudate.

In another embodiment, the article of the present invention is used for wound healing.

In another aspect, the article of the present invention is used as an anti-adhesive membrane in surgical devices.

In another embodiment, the present invention relates to a method of promoting the wound healing comprising contacting the wound with said article optionally containing the bioactive compound.

In yet another embodiment, the present invention discloses a non-adherent, absorbent article that has optimum MVTR and hence facilitates accelerated healing of wounds when used for wound dressing.

In another embodiment, the non-adhesive, absorbent article of the present invention acts as an anti-adhesive membrane with desired properties with drugs incorporated into the membrane, so that the drug can be delivered locally over a period of time to the surgical site.

The non-adherent, absorbent article of the present invention has good handling characteristics when used for wound dressings as well as during a surgical procedure, is compatible to the tissues, is resorbable and possesses sufficient bio adhesiveness to ensure secure placement at the wound and/or surgical site until the likelihood of adhesion formation is minimized.

In an embodiment, the present invention provides a method of promoting the wound healing comprising contacting the wound with the article optionally containing the bioactive compound.

In another embodiment, the present invention relates to the use of non-adherent absorbent article to heal any type of skin injuries.

In another embodiment, the non-adherent absorbent article of the present invention containing the bioactive molecule provides controlled release of the molecule based on the treatment condition.

In yet another embodiment the present invention relates to a non-adherent, absorbent wound dressing composition with optimum moisture vapor transmission rate (MVTR) comprising;
iii. Regenerated SF along with Hydrophilic synthetic and/or natural polymer coated/deposited on to the mesh; and
iv. optionally loaded with bioactive molecules in an amount of 0.01 to 5 mg/ml;
   wherein said SF to hydrophilic polymer is in the ratio ranging between 95:05 to 50:50.

The concentration of regenerated SF is in range of 0.25-8 wt %; preferably 1-4 wt %.

The Hydrophilic synthetic and/or natural polymer is in the concentration ranging between 0.25-8 wt %; preferably 1-4 wt %; the silk content in regenerated silk fibroin in the concentration of 65-95%.

The hydrophilic synthetic polymer is water soluble polymer selected from polyethylene glycol, polyethylene oxide, polyvinyl alcohol and the like alone or in combination thereof.

The hydrophilic natural polymer is water soluble polymer selected from collagen, chitosan, alginate, sericin, hyaluronic acid and the like alone or in combination thereof.

The bioactive molecule is selected from pharmaceutical drugs, nanoparticles, growth factors, healing agents, disinfectants, astringents, regenerating agents and such like alone or combinations thereof in an amount of 0.01 to 5 mg/ml.

In an embodiment, the present invention provides non-adherent, absorbent article and to the composition thereof that exhibits improved MVTR, water absorption, improved tear resistance, is painless and also enables loading of the bioactive molecules.

### EXPERIMENTAL

PEO from Sigma Aldrich,
Chitosan from Sigma Aldrich
PVA from Laboratory Rasayan (S. D. Fine-Chem Ltd.)

### Examples: General Process for Fabrication of Non-Adherent, Absorbent Article

### Example 1: Silk Fibroin Solution Preparation

Bombyx mori pure bivoltine cocoons were boiled in 0.5 w/v % of NaHCO₃ solution twice for 30 min each for sericin removal. Collected fibroin was vacuum dried at 60° C. followed by dissolution in 9.3M LiBr at 60° C. for 4 h. This solution was dialyzed extensively against water for 48 h to obtain regenerated Silk Fibroin (SF) solution with 5-6 wt % concentration. This solution was diluted to obtain 1 to 4 wt % solutions by using deionized water (DI) to obtain various compositions.

### Example 2: Polymer Solution and Blend Preparation

Desired concentrations of aqueous solutions of either synthetic or natural polymers were prepared by dissolving the hydrophilic polymer in DI water at room temperature. The hydrophilic polymer solution was mixed with the same concentration of SF solutions in volume ratios ranging between 95:05 to 50:50. Further, the polymer solution was poured into the mold in which a mesh was kept. The samples were frozen at −80° C. and lyophilized for 12-18 h followed by exposure of the samples to methanol. The exposure to methanol enabled the conformation transition in SF protein and improved their stability.

### Example 3: Evaluation of Desired Properties

### (A) Water Absorption

The percentage water absorption was studied by using the following protocol. The sample was cut into 1 cm×1 cm size piece and the initial weight (W1) of the sample was recorded. The sample was immersed in 2 ml of DI solution. After 2 h, the sample was removed and the final weight (W2) was recorded after gently tapping on tissue paper to remove excess dripping water. % water absorption was calculated by using following formula.

$$\% Water absorption = \frac{W 2 - W 1}{W 1} \times 100$$

It has been observed from the FIG. 2 that % water absorption is higher when lower polymer solution concentration is used. The absorption was found to be higher for polymer concentrations between 1-3%, while as the concentration of polymer was increased to 4%, the absorption was found to be very low.

Below mentioned scale system has been used to compare between the different formulation.

| % Absorption at | |
|---|---|
| the end of 2 h | Scale |
| >1000 | ++++ |
| >500 | +++ |
| >300 | ++ |
| >100 | + |

When the percentage absorption was studied with different mixing ratios it has been observed that percentage absorption is higher for any hydrophilic polymer, where ratio of SF:HP is 75:25 (FIG. 3A).

With reference to FIG. 3B, with increase in thickness of cotton gauze (CG), the percent absorbance was observed to decrease.

### (B) Moisture Vapor Transmission Rate (MVTR)

MVTR is an important property that governs the performance of an advance wound care dressing. To measure the MVTR of developed wound dressings, a Paddington cup was used. The cup contains a mold with a holding capacity of 15-18 mL liquid and a lid. Weight of the assembly together with the base and lid was recorded (W1). Samples were kept on the surface in such a way that the sample must not come in contact with DI and tightly covered with upper closing lid of MVTR assemblies. Approximately 15 ml DI was added in the mold and the weight of the assembly was recorded (W2).

The assembly was placed in the incubator at 37° C. for 24 h. At the end of incubation, the assembly was removed and kept in the desiccator and allowed to equilibrate at room temperature for 30 minutes. Post this, the weight was recorded (W3). Further, the assembly was also reweighed after removing all the water (Assembly without water+Sample+lid) (W4). Calculate the mass of moisture vapor lost through the dressings (W2-W3).

Calculate MVTR (g/m²/24 h)=(W2-W3)/Area of sample. As MVTR is highly influenced by thickness of the sample, resultant MVTR has been normalized with thickness and reported as g/m²/24 h/cm.

Observations: From the results (FIG. 4A) it has been observed that addition of hydrophilic polymer to SF results in significant increase in MVTR. Below mentioned scale system has been used to compare between the different concentrations.

| MVTR | Scale |
|---|---|
| >3000 | ++++ |
| >2000 | +++ |
| >1000 | ++ |
| >500 | + |

Further, FIG. 4B depicts the Normalized MVTR with various layer of cotton gauze (CG). It was observed that the MTVR rate increases with increase in layers of CG with silk blended with hydrophilic polymer.

The FIG. 4C depicts the Normalized MVTR with different woven and/or non-woven silk mesh blended with hydrophilic polymer.

### (C) Non-Adherent Property

Non-adherent wound dressings are highly desirable as they reduce discomfort and pain for patient during dressing change. Further, non-adherent dressings also facilitate superior and faster healing because removal of dressing does not disturb the top healing surface. However, there are no standard protocols for testing non-adherence of dressings in in-vitro tests. The present inventors have developed and standardized a protocol to establish non-adherence.

A single blood drop was taken by finger prick method and placed on a clean glass slide. Immediately after collecting the bleed drop, the developed article was kept on the glass slide in such a way that blood drop was covered completely. The samples were incubated for 3 h. After 3 h, the samples were peeled off from the surface of the glass slide as depicted in FIG. 1. The fig confirms the non-adherence property of said article which could be used for wound dressing.

### Example 4: Process to Load Bioactive Molecules

Vancomycin at the concentration of 0.5 mg/mL was loaded in the polymer solutions while preparing the article for wound dressing. A 1 cm×1 cm piece of said article for wound dressing prepared as per protocol described in Example 1 and Example 2 was used to test the release of the drug molecule. The piece was incubated in DI water 25° C. for 24 h. Release in DI was measured by UV spectroscopy by recording the absorbance at 280 nm. The absorbance value was used to calculate the actual concentration of drug released by comparing the absorbance with the standard curve.

As can be seen from the FIG. 5, the drug can be successfully incorporated and released from said article used for wound dressing. This proves that the process is flexible enough to incorporate various active biomolecules, drugs, growth factors, etc. into the article.

### Example 5: Summary of the Properties of Different Compositions of Said Article

| | Different Compositions | | | | | | | |
|---|---|---|---|---|---|---|---|---|
| | | | 1CG + | | | 1CG + | 1CG + | |
| | | | 2% SF | (1CG + | 1CG + | 2% SF | 4% SF | 1CG + |
| | Cotton | | (50%) + | 2% SF | 4% SF | (75%) + | (50%) + | 2% SF |
| | Gauze | 1 CG + | 2% | (75%) + | (75%) + | 2% | 4% | (75%) + |
| | (16 | 2% | PEO | 2% PEO | 4% PEO | CS | CS | 2% PVA |
| Properties | ply) | SF | (50%) | (25%) | (25%) | (25%) | (50%) | (25%) |
| MVTR | +++ | ++ | +++ | ++++ | +++ | ++++ | ++++ | ++++ |
| (g/m2/24 h/cm) | | | | | | | | |
| % absorption | +++ | ++++ | ++ | ++++ | ++ | ++++ | ++++ | ++++ |
| (2 h) | | | | | | | | |
| Non-adherent | X | X | ✓ | ✓ | X | ✓ | X | ✓ |

### Example 6: In-Vitro Wound Healing Study

### Animals:

15-16 weeks old Wistar rats weighing 289 g to 427 g were used for the study. The animal room was supplied with fresh and filtered air, with 10-15 air changes per hour. The room temperature was set at 17-25° C. with 50-70% relative humidity and illumination cycle was set to 12 h of light and 12 h dark. All the animals were given free access to standard food and water ad-libitum.

Wound creation: Animals were shaved 24 h before the wound creation. On the day of wound creation animals were subjected to general anaesthesia (xylazine (10 mg/kg) and ketamine (90 mg/kg). Circular full thickness wounds of 10 mm diameter were created on both sides of midline using 10 mm skin biopsy punch. The control wounds were untreated and left open without any dressing. Cotton gauze and SF wound dressing (Example 3) were applied on to each wound ensuring the wound edges are covered with the dressing. Dressing was secured using breathable micropore tape. Dressing was changed every 3^{rd} day for 15 days. Wounds were photographed during every dressing change.

### Results:

All the experimental groups showed 100% survival. Wound closure panel is shown in the FIG. 6. FIG. 6A-C represent wound photographs on the day of wound creation (Day 0) while FIG. 6D-F represent the photographs of wounds on Day 15.

At Day 15, silk wound dressing showed significantly higher wound closure (98.9±0.3%) than untreated wounds (96.4±0.3%) and cotton gauze treated (92.3±0.7%) wounds.

It will be understood that the above description is intended to be illustrative and not restrictive. The embodiments will be apparent to those in the art upon reviewing the above description. The scope of the invention should therefore, be determined not with reference to the above description but should instead be determined by the appended claims along with full scope of equivalents to which such claims are entitled.

1. A non-adherent, absorbent article with optimum moisture vapor transmission rate (MVTR) comprising;
i. a first contact layer of regenerated silk fibroin (SF) containing a hydrophilic natural polymer and/or a hydrophilic synthetic polymer or a combination thereof coated/deposited on to a mesh; and
ii. optionally loaded with a bioactive molecules,
wherein the SF to the hydrophilic natural polymer and/or the hydrophilic synthetic polymer or a combination thereof are in the ratio ranging between 95:05 to 50:50.

2. The article as claimed in claim 1, wherein the article has a thickness of 1-20 mm and a density of 0.02-0.08 g/cm³.

3. The article as claimed in claim 1, wherein (a) the regenerated silk fibroin (SF) is in a concentration ranging between 0.25-8 wt %; (b) the hydrophilic natural polymer and/or the hydrophilic synthetic polymer or a combination thereof is in a concentration ranging between 0.25-8 wt %.

4. The article as claimed in claim 3, wherein the hydrophilic synthetic polymer is a water soluble polymer comprising polyethylene glycol, polyethylene oxide, polyvinyl alcohol or a combination thereof.

5. The article as claimed in claim 3, wherein the hydrophilic natural polymer is a water soluble polymer comprising collagen, chitosan, alginate, sericin, hyaluronic acid or a combination thereof.

6. The article as claimed in claim 1, wherein the bioactive molecule comprises a pharmaceutical drug, a nanoparticle, a growth factor, a healing agent, a disinfectant, an astringent, a regenerating agent, a natural plant/herbal extract, an ion, a phytochemical or a combination thereof in a concentration of 0.01 to 5 mg/ml.

7. The article as claimed in claim 1, wherein the mesh is a woven mesh and/or a non-woven mesh.

8. The article as claimed in claim 1, wherein the article can be removed physically without any pain and without disturbing a top healing surface.

9. The non-adherent, absorbent article with optimum MVTR as claimed in claim 1, comprising;
i. a regenerated SF solution in a concentration of 2 wt % along with the hydrophilic natural polymer comprising chitosan in a concentration of 2 wt %; coated/deposited on to the mesh; and
ii. optionally loaded with a bioactive molecule in an amount of 0.01 to 5 mg/ml;
wherein the SF to chitosan is in a weight ratio of 75:25.

10. The non-adherent, absorbent article with optimum MVTR as claimed in claim 1, comprising;
i. a regenerated SF solution in a concentration of 2 wt % along with the hydrophilic synthetic polymer comprising polyethylene oxide (PEO) in a concentration of 2 wt % coated/deposited on to the mesh; and
ii. optionally loaded with bioactive molecules in an amount of 0.01 to 5 mg/ml;
wherein the SF to PEO is in a weight ratio of 75:25.

11. A process for fabricating non-adherent, absorbent article with optimum MVTR comprising;
i. preparing the regenerated silk fibroin solution;
ii. mixing the regenerated SF solution with the hydrophilic synthetic polymer and/or the hydrophilic natural polymer or a combination thereof in a weight ratio ranging between 95:05 to 50:50 to obtain a polymer solution;
iii. placing the mesh into a mold;
iv. pouring a required amount of the polymer solution of step (ii) in to the mold and freezing, followed by lyophilization;
v. annealing the lyophilized product with lower alcohol.

12. The process as claimed in claim 11, wherein to the process step (ii) is optionally added the bioactive molecule selected from a pharmaceutical drug, a nanoparticle, a growth factor, a healing agent, a disinfectant, an astringent, a regenerating agent, a natural plant/herbal extract, an ion, a phytochemical or a combinations thereof in a concentration of 0.01 to 5 mg/ml.

13. The process as claimed in claim 11, wherein a silk content in the regenerated silk fibroin is 65-95%.

14. The process as claimed in claim 11, wherein the hydrophilic synthetic polymer is a water soluble polymer comprising polyethylene glycol, polyethylene oxide, polyvinyl alcohol or in a combination thereof in a concentration ranging between 0.25-8.0 wt.

15. The process as claimed in claim 11, wherein the hydrophilic natural polymer is a water soluble polymer comprising collagen, chitosan, alginate, sericin, hyaluronic acid or in a combination thereof in a concentration ranging between 0.25-8.0 wt %.

16. A non-adherent, absorbent wound dressing composition with optimum moisture vapor transmission rate (MVTR) comprising;
i. a regenerated SF along with a hydrophilic synthetic polymer and/or a hydrophilic natural polymer or a combination thereof coated/deposited on to a mesh; and
ii. optionally loaded with a bioactive molecule in an amount of 0.01 to 5 mg/ml;
wherein the SF to the hydrophilic synthetic polymer and/or a hydrophilic natural polymer or a combination thereof is in a weight ratio ranging between 95:05 to 50:50.

17. The wound dressing composition as claimed in claim 16, wherein (i) a concentration of the regenerated SF is in a concentration ranging between 0.25-8 wt %; (ii) the hydrophilic synthetic polymer and/or the hydrophilic natural polymer or a combination thereof is in a concentration ranging between 0.25-8 wt %.

18. (canceled)

19. The wound dressing composition as claimed in claim 16, wherein a silk content in the regenerated silk fibroin is 65-95%.

20. The wound dressing composition as claimed in claim 19, wherein (i) the hydrophilic synthetic polymer is a water soluble polymer comprising polyethylene glycol, polyethylene oxide, polyvinyl alcohol or in a combination thereof; (ii) the hydrophilic natural polymer is a water soluble polymer comprising collagen, chitosan, alginate, sericin, hyaluronic acid or in a combination thereof; (ii) the bioactive molecule comprises a pharmaceutical drug, a nanoparticle, a growth factor, a healing agent, a disinfectant, an astringent, a regenerating agent or a combination thereof in an amount of 0.01 to 5 mg/ml.

21. (canceled)

22. (canceled)

23. (canceled)

24. (canceled)

25. A method of promoting a wound healing comprising contacting a wound with the non-adherent, absorbent article as claimed in claim 1.
